(19) United States
(12) Patent Application Publication
(10) Pub. No.: US 2008/0146898 A1
(43) Pub. Date: Jun. 19, 2008

(54) SPECTRAL WINDOWS FOR SURGICAL TREATMENT THROUGH INTERVENING FLUIDS

(75) Inventors: Michael P. Weir, Blanchester, OH (US); Robert J. Dunki-Jacobs, Mason, OH (US)

Correspondence Address: Mark P. Levy;Thompson Hine LLP, P.O. Box 8801, Dayton, OH, 45401-8801, US

(73) Assignee: ETHICON ENDO-SURGERY, INC., Cincinnati, OH (US)

(21) Appl. No.: 11/612,686

(22) Filed: Dec. 19, 2006

(51) Int. Cl. A61B 6/00

(52) U.S. Cl. 600/310

(57) ABSTRACT

A scanning beam assembly for use in medical applications comprising a plurality of radiation emitters that emit radiation over different wavelength ranges, wherein at least one of the emitters emits radiation that is minimally absorbed by blood; a scanner including a reflector that receives radiation from the emitters and directs it onto a field-of-view; and at least one detector configured to receive the radiation scattered, reflected or transmitted by the field-of-view and generate an electrical signal.

### FIELD OF THE INVENTION

The present invention is related generally to medical devices adapted for treatment, and more particularly to a medical device using a scanned beam assembly to treat and/or image body tissue.

### BACKGROUND OF THE INVENTION

U.S. Published Application 2005/0020926A1 discloses a scanning beam imager 102 which is reproduced in FIG. 1 herein. This imager 102 can be used in applications in which cameras have been used in the past. In particular it can be used in medical devices such as video endoscopes, laparoscopes, etc. Illuminator 104 may include multiple emitters such as, for instance, light emitting diodes (LEDs), lasers, thermal sources, arc sources, fluorescent sources, gas discharge sources, or other types of illuminators. In some embodiments, illuminator 104 comprises a red laser diode having a wavelength of approximately 635 to 670 nanometers (nm). In another embodiment, illuminator 104 comprises three lasers: a red diode laser, a green diode-pumped solid state (DPSS) laser, and a blue DPSS laser at approximately 635 nm, 532 nm, and 473 nm, respectively. Illuminator 104 may include, in the case of multiple emitters, beam combining optics to combine some or all of the emitters into a single beam. Illuminator 104 may also include beam-shaping optics such as one or more collimating lenses and/or apertures. Additionally, while the wavelengths described in the previous embodiments have been in the optically visible range, other wavelengths may be within the scope of the invention. Light beam 106, while illustrated as a single beam, may comprise a plurality of beams converging on a single scanner 108 or onto separate scanners 108.

The illumination sources disclosed in U.S. Patent Application Publication 2005/0020926A1 suffer from drawbacks that limit their utility in surgical practice. Blood vessels may be deliberately or accidentally cut or injured during surgical procedures. The resulting flow of blood often collects in a pool or film, which obscures the source of the blood, until the compromised vessel is clamped or ligated in order to prevent further flow. It would be an advantage for the surgeon to be able to see through the pool or film of blood to observe body tissue and/or to effect medical or surgical treatment.

Accordingly, there is a need for imagers using auxiliary illumination and detectors sensitive to wavelengths allowing visibility through blood, thereby increasing the quality of the image or view obtained during a particular surgical procedure.

### SUMMARY OF THE INVENTION

One embodiment of the invention is a scanning beam assembly for use in medical applications, comprising a plurality of radiation emitters that emit radiation over different wavelength ranges, wherein at least one of the emitters emits radiation that is minimally absorbed by blood, a scanner including a reflector that receives radiation from the emitters and directs it onto a field-of-view, and at least one detector configured to receive the radiation scattered, reflected, or transmitted by the field-of-view and generate an electrical signal.

Another embodiment of the present invention is a method for viewing body tissue in the presence of blood comprising the steps of (a) providing a medical device including a plurality of radiation emitters that emit radiation over different wavelength ranges coupled into an optical fiber assembly having at least one signal inlet, at least one of the emitters operating in a wavelength range that is minimally absorbed by blood, a scanner including at least one reflector configured to direct the radiation from the emitters onto a field-of view, at least one detector configured to receive and detect the radiation scattered, reflected, or transmitted by the surrounding field-of-view, (b) generating a video image stream based on electrical signals generated by the detector(s), and (c) displaying a video image of the field-of-view to a user. The term “viewing” as used herein does not require the formation of an image. It includes procedures in which a tissue may be examined optically, electronically, or otherwise and treated accordingly.

The present invention has, without limitation, application in conventional endoscopic, laparoscopic, and open surgical instrumentation as well as application in robotic-assisted surgery.

The details of one or more embodiments of the invention are set forth in the accompanying drawings and the description below. Other features, objects and advantages of the invention will be apparent from the description and the drawings, and from the claims.

### BRIEF DESCRIPTION OF THE FIGURES

FIG. 1 is a block diagram of the scanning beam imager disclosed in U.S. Published Application 2005/0020926A1.

FIG. 2 is a schematic illustration of a medical device system adapted for imaging including a scanned beam unit, constructed in accordance with one embodiment of the present invention; and

FIG. 3 is a schematic illustration showing a radiation source including multiple emitters for generating imaging and/or diagnostic beams of radiation, constructed in accordance with one embodiment of the present invention.

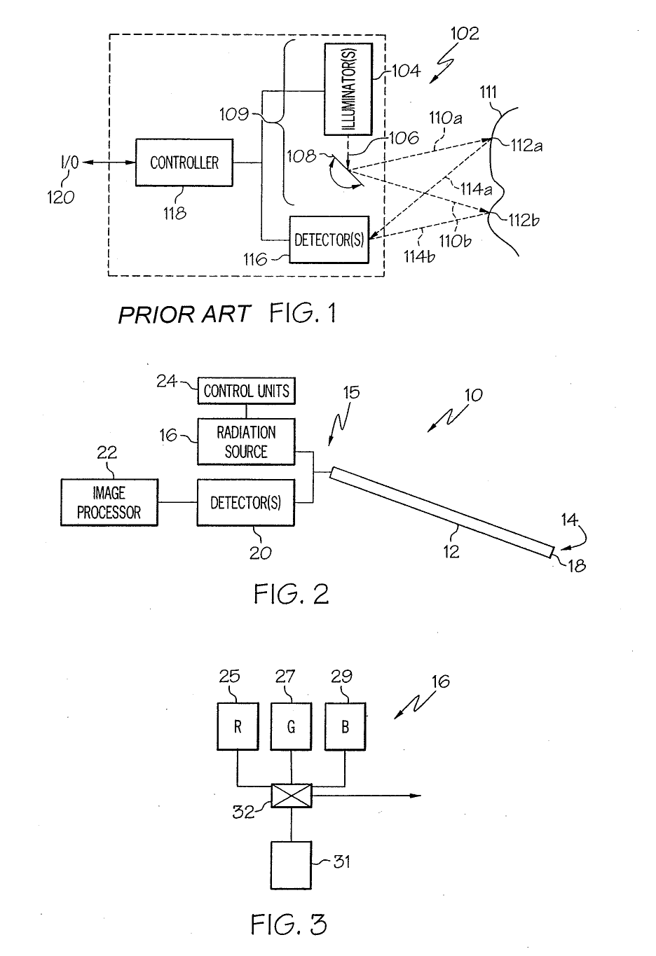

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

Referring to FIG. 2, a medical device 10 includes an endoscope generally including an elongate, flexible or rigid tube 12 having a distal end 14 and a proximal end 15 opposite the distal end 14. As used herein, an endoscope 12 refers to an instrument for use in examining, treating and/or diagnosing the interior of a body cavity. As used herein, the term “proximal” refers to a location on the medical device 10 or a component thereof that is closer to the user or physician and the term “distal” refers to a location on the medical device 10 or a component thereof that is further from the user or physician and closer to the operative site. Typically, a source of radiation 16 of the medical device 10 is located outside a patient's body and at least the distal end 14 of the medical device 10 is insertable into the patient's body for a surgical procedure. Furthermore, while an endoscope 12 is referred to, any other suitable type of medical device may be used, for example, any medical catheter or any medical scope such as but not limited to a gastroscopes, enteroscopes, sigmoidscopes, colonoscopes, laryngoscopes, bronchoscopes, duodenoscopes, cystoscopes, hysteroscopes, arthroscopes.

In one embodiment of the present invention, as shown in FIG. 2, a medical device 10 includes a scanned beam unit 18 that is capable of directing radiation from the radiation source 16 onto a surface, such as tissue on or within a patient's body. In some instances, radiation reflected from the surface is collected and directed back through the endoscope 12 to one or more photodetectors 20. One or more photodetectors 20 receive the radiation and produces electrical signals corresponding to the amount of radiation received. The signals can be used by an image processor 22 to generate a digital image, e.g., for processing, decoding, archiving, printing, display, et cetera.

Radiation source 16 may include multiple emitters such as, for instance, radiation emitting diodes (LED's), lasers, thermal sources, arc sources, fluorescent sources, gas discharge sources, or others. Radiation source 16 may be tunable using control unit 24. In some embodiments, radiation source 16 is capable of providing multiple types of radiation, for example, selected for imaging, therapy, diagnosis, or combinations thereof.

FIG. 3 illustrates that radiation source 16 may include multiple emitters 25, 27, 29 for generating imaging, therapeutic, and/or diagnostic beams of radiation, each emitter 25, 27, 29 may be capable of generating radiation at a predetermined wavelength. Beam combiner 32 combines the radiation from the multiple emitters 25, 27, 29 into a single beam. The multiple emitters may be controllable using control unit 24, shown in FIG. 2. In one embodiment, emitter 25 is capable of generating a red beam of radiation, emitter 27 is capable of generating a green beam of radiation and emitter 29 is capable of generating a blue beam of radiation. In one embodiment, the red, green and blue beams of radiation may have wavelengths of approximately 635 nm, 532 nm, and 473 nm. In another embodiment, at least one of the multiple emitters 25, 27, 29 is capable of generating a beam of radiation having a wavelength in the spectral window that is minimally absorbed by blood.

In one embodiment, radiation source 16 includes an auxiliary emitter 31 that is capable of generating a beam of radiation having a wavelength in the spectral window that is minimally absorbed by blood, as will be described in greater detail below. In another embodiment, auxiliary emitter 31 may generate a beam of radiation that is minimally absorbed by blood and function as a therapeutic beam. Beam combiner 32 may combine the radiation from auxiliary emitter 31 with the radiation from the multiple emitters 25, 27, 29 into a single beam. In another embodiment, the radiation from auxiliary emitter 31 is a separate beam.

Blood within the FOV interacts with incoming radiation from the radiation source 16, reflected radiation from the surfaces within the FOV, and any other radiation source present by absorbing, transmitting, reflecting, and scattering the radiation. For visible wavelengths, absorption by a blood film or blood pool reduces the amount of illumination reaching the underlying tissue(s) or surfaces of the body and further reduces the amount of reflected radiation reaching the detector. Auxiliary emitter 31 emits radiation in the spectral windows where the radiation is minimally absorbed by blood at an intensity such that upon detection by photodetector(s) 20 and processing by image processor 22 enhances the quality of the image obtained. Since the transmittance of radiation in the spectral window is not high, in one embodiment, the emitter 31 may operate at an intensity that is at least about ten times the intensity of the visible radiation emitters. In another embodiment, the emitter 31 may operate at an intensity that is at least about 50 times the intensity of the visible radiation emitters.

Based on the oxygenation level of the blood pool, the clinician may select from a plurality of the spectral windows at which to operate the emitter 31. Compared to human medicine, veterinary medicine applications may require a different complement of wavelength choices and selection methodology. The clinician should select the spectral window that minimizes absorption of the radiation by the blood. Control unit 24 enables the clinician to select the spectral window that is most appropriate for the particular circumstances. In one embodiment, control unit 24 switches between various emitter sources that are set at specific wavelengths. In another embodiment, control unit 24 filters the wavelengths emitted from an emitter source to only allow the selected spectral window of wavelengths or even a single wavelength to be received by the scanner.

Examples are provided below in which the particular subject's blood is either human or porcine, and the blood is either oxygenated or deoxygenated. Many other spectral windows are possible for other animal related blood applications. For example, oxygenated porcine blood exhibits minimal absorption of radiation between spectral windows at about 650-750 nm, about 1050-1150 nm, and about 1200-1300 nm, and deoxygenated porcine blood exhibits minimal absorption of radiation between spectral windows at about 650-750 nm and about 1250-1300 nm. As another example, oxygenated human blood exhibits minimal absorption of radiation between spectral window at about 650-750 nm and about 1050-1150 nm. Deoxygenated human blood exhibits minimal absorption of radiation between spectral window at about 700-750 nm and about 1050-1150 nm.

Additionally, U.S. Pat. No. 6,178,346 discloses that visualization through opaque-body-fluid environments, such as blood, is improved by using wavelengths in the infrared. The patent discloses that low scattering by the suspended cells and low absorption by water and hemoglobin can be obtained in the wavelength regions: 1400-1800 nm, 2100-2400 nm, 3700-4300 nm, 4600-5400 nm, and 7000-14000 nm. In still another embodiment, the spectral window includes radiation in the range of about 1500-1800 nm. It is noted that in describing the auxiliary emitter with respect to the wavelength range, it is only necessary that the emitter emit radiation at one or more wavelengths within the ranges as opposed to the entire range. The radiation emitter 31 may be an illumination source, that emits over a wavelength range, including one or more wavelengths in the ranges of 650-750 nm, 1050-1300 nm, 1400-1800 nm, and 2100-2400 nm. High power radiation sources that emit in these ranges are commercially available.

Furthermore, at least one or more of the photodetectors 20 must absorb effectively within the aforesaid ranges. Photodetectors that are sensitive to radiation in the spectral window are commercially available. Additionally, the optical fibers employed in the scanners must be able to transmit the radiation and particularly the auxiliary radiation. One type of optical fiber that is useful for transmitting infrared radiation is a so-called holey optical fiber.

In one embodiment, the image acquired in the auxiliary radiation (long wavelength) detector channel may be overlaid on the full-color image obtained from the image signal acquired from the visible radiation detector channels. In another embodiment, the image acquired from the auxiliary radiation detector channel may be overlaid in a false color, for example, one not often seen in normal anatomy. In summary, the image obtained from the auxiliary signal may replace the full color image, or be added in so as to preserve anatomical detail.

In one aspect of the present invention, a method for viewing body tissue in the presence of blood includes the steps of: a) providing a medical device including a plurality of radiation emitters that emit radiation over different wavelength ranges coupled into an optical fiber assembly having at least one signal inlet, at least one of the emitters operating in a wavelength range that is minimally absorbed by blood, a scanner including at least one reflector configured to direct the radiation from the emitters onto a field-of view, and at least one detector configured to receive and detect the radiation scattered, reflected, or transmitted by the surrounding field-of-view; b) generating a video image stream based on electrical signals generated by the detector(s); and c) displaying a video image of the field-of-view to a user.

In accordance with other embodiments of the invention, an auxiliary emitter 31 emitting radiation minimally absorbed by blood and, more particularly, within one or more of the wavelength ranges disclosed herein can be used in conjunction with one or more of the scanning beam imagers described in U.S. Pat. No. 7,071,594 and U.S. Published Application 2005/0116038, both assigned to Microvision, Inc.

The foregoing description of several embodiments and expressions of the invention have been presented for purposes of illustration. It is not intended to be exhaustive or to limit the invention to the precise forms disclosed, and obviously many modifications and variations are possible in the above teaching. For example, as would be apparent to those skilled in the art, the disclosures herein of the medical device for imaging have equal application in robotic assisted surgery taking into account the obvious modifications of such systems and components to be compatible with such a robotic system.

1. A scanning beam assembly for use in medical applications, comprising:
a plurality of radiation emitters that emit radiation over different wavelength ranges, wherein at least one of the emitters emits radiation that is minimally absorbed by blood;
a scanner including a reflector that receives radiation from the emitters and directs it onto a field-of-view; and
at least one detector configured to receive the radiation scattered, reflected, or transmitted by the field-of-view and generate an electrical signal.

2. The assembly of claim 1 further comprising an image processor that generates a video image stream based on electrical signals generated by the detector.

3. The assembly of claim 2 further comprising a display device for displaying a video image of the field-of-view to a user.

4. The assembly of claim 1, wherein the radiation includes radiation in the range of about 650-2400 nm.

5. The assembly of claim 1, wherein the radiation includes radiation in the range of about 750-1700 nm.

6. The assembly of claim 1, wherein the blood is oxygenated human blood.

7. The assembly of claim 6, wherein the radiation includes radiation within the range of 650-1150 nm.

8. The assembly of claim 6, wherein the radiation includes radiation within the range of 650-750 nm.

9. The assembly of claim 6, wherein the radiation includes radiation within the range of 1050-1150 nm.

10. The assembly of claim 1, wherein the blood is deoxygenated human blood.

11. The assembly of claim 10, wherein the radiation includes radiation within the range of 700-1150 nm.

12. The assembly of claim 10, wherein the radiation includes radiation within the range of 700-750 nm.

13. The assembly of claim 10, wherein the radiation includes radiation within the range of 1050-1150 nm.

14. The assembly of claim 1, wherein the blood is porcine blood.

15. The assembly of claim 14, wherein the porcine blood is oxygenated.

16. The assembly of claim 1, wherein the radiation that is minimally absorbed by blood includes radiation in the range of about 1400-1800 nm.

17. The assembly of claim 1, wherein the radiation that is minimally absorbed by blood includes radiation in the range of about 2100-2400 nm.

18. A method for viewing body tissue in the presence of blood comprising the steps of:
a) providing a medical device including
(i) a plurality of radiation emitters that emit radiation over different wavelength ranges coupled into an optical fiber assembly having at least one signal inlet,
(ii) at least one of the emitters operating in a wavelength range that is minimally absorbed by blood,
(iii) a scanner including at least one reflector configured to direct the radiation from the emitters onto a field-of view,
(iv) at least one detector configured to receive and detect the radiation scattered, reflected, or transmitted by the surrounding field-of-view;
b) generating a video image stream based on electrical signals generated by the detector(s); and
c) displaying a video image of the field-of-view to a user.

19. The method of claim 18, wherein the radiation includes radiation in the range of about 650-2400 nm.

20. The method of claim 18, wherein the radiation includes radiation in the range of about 750-1700 nm.

21. The method of claim 18, wherein the blood is oxygenated human blood.

22. The method of claim 21, wherein the radiation includes radiation within the range of 650-1150 nm.

23. The method of claim 21, wherein the radiation includes radiation within the range of 650-750 nm.

24. The method of claim 21, wherein the radiation includes radiation within the range of 1050-1150 nm.

25. The method of claim 18, wherein the blood is deoxygenated human blood.

26. The method of claim 25, wherein the radiation includes radiation within the range of 700-1150 nm.

27. The method of claim 25, wherein the radiation includes radiation within the range of 700-750 nm.

28. The method of claim 25, wherein the radiation includes radiation within the range of 1050-1150 nm.

29. The method of claim 18, wherein the blood is porcine blood.
